(19) United States
(12) Patent Application Publication
(10) Pub. No.: US 2024/0042439 A1
(43) Pub. Date: Feb. 8, 2024

(54) CHIP AND METHOD FOR ISOLATING RED BLOOD CELL FROM WHOLE BLOOD

(71) Applicant: National Tsing Hua University, Hsinchu City (TW)

(72) Inventors: Fan-Gang Tseng, Hsinchu City (TW); Tsung-Han Ho, Hsinchu City (TW); Cian-Ling Wang, Hsinchu City (TW)

(73) Assignee: National Tsing Hua University, Hsinchu City (TW)

(21) Appl. No.: 18/182,362

(22) Filed: Mar. 13, 2023

(30) Foreign Application Priority Data
Aug. 4, 2022 (TW) 111129424

(51) Int. Cl. B01L 3/00

(52) U.S. Cl. CPC B01L 3/502753

(57) ABSTRACT

In an embodiment, a chip is used for isolating RBC from a whole blood and includes an accommodating space, a porous membrane and a microchannel system. The accommodating space is configured for containing a whole blood sample including RBC debris. The porous membrane is disposed at a bottom of the accommodating space and includes a plurality of pores. A diameter of the pores is larger than a size of the RBC debris and larger than a mean diameter of WBCs and CTCs. The microchannel system includes a liquid inlet, a liquid outlet and a microchannel. The microchannel is disposed between and communicates with the liquid inlet and the liquid outlet. The microchannel is disposed under the porous membrane.

### CROSS-REFERENCE TO RELATED APPLICATION

This application claims the priority benefit of Taiwan application serial no. 111129424, filed on Aug. 4, 2022. The entirety of the above-mentioned patent application is hereby incorporated by reference herein and made a part of this specification.

### BACKGROUND

### Technical Field

The invention relates to a chip and method, and in particular, relates to a chip and method for isolating red blood cells from a whole blood.

### Description of Related Art

Currently, when circulating tumor cells (CTCs) are detected, a pre-treatment is performed by a centrifuge. Specifically, blood cells having different densities in the blood sample are separated by using centrifugal force, so as to isolate red blood cells and obtain peripheral blood mononuclear cells (PBMC). Then, a detection such as a cell staining may be performed. However, since CTCs are rare cells, it is difficult to ensure that all CTCs are retained by obtaining PBMCs through centrifugation. In addition, the centrifugal force may damage cell activity of CTCs. Therefore, there is an urgent need in the art for a method that may isolate the red blood cells without using centrifugation, and avoid loss and impaired activity of leukocytes, CTCs and other cells.

SUMMARY

The invention provides a chip for isolating red blood cells from a whole blood.

The invention further provides a method for isolating red blood cells from a whole blood.

In an embodiment, a chip is used for isolating RBC from a whole blood and includes an accommodating space, a porous membrane and a microchannel system. The accommodating space is configured for containing a whole blood sample including RBC debris. The porous membrane is disposed at a bottom of the accommodating space and includes a plurality of pores. A diameter of the pores is larger than a size of the RBC debris and larger than a mean diameter of WBCs and CTCs. The microchannel system includes a liquid inlet, a liquid outlet and a microchannel. The microchannel is disposed between and communicates with the liquid inlet and the liquid outlet. The microchannel is disposed under the porous membrane.

In an embodiment, a material of the porous membrane includes polyethylene terephthalate (PET).

In an embodiment, the pores of the porous membrane are regularly arranged.

In an embodiment, the diameter of the pores of the porous membrane is less than 8 um.

In an embodiment, a liquid flowing through the microchannel generates a vertical flow field, and the vertical flow field drives the RBC debris to penetrate through the porous membrane into the microchannel.

In an embodiment, the microchannel system further includes a pump, and the vertical flow field is generated by causing a flow rate difference between a liquid entering the liquid inlet and the liquid exiting the liquid outlet through the pump.

In an embodiment, a method for isolating red blood cells from a whole blood includes the following steps. The chip is provided. A blood sample is mixed with a red blood cell lysing solution, to lyse red blood cells into RBC debris. The blood sample including the RBC debris is dropped into the accommodating space of the chip. A washing liquid is provided to the microchannel system of the chip, so that a flow rate difference between the washing liquid entering the liquid inlet and the washing liquid exiting the liquid outlet is generated, and the flow rate difference drives the RBC debris in the blood sample to penetrate through the porous membrane into the microchannel.

In an embodiment, the flow rate difference between the washing liquid entering the liquid inlet and the washing liquid exiting the liquid outlet is greater than or equal to 50 ml/hour.

In an embodiment, the diameter of the pores of the porous membrane is less than 8 um.

In an embodiment, after driving the RBC debris in the blood sample to penetrate through the porous membrane into the microchannel, WBCs and CTCs in the blood sample are retained on the porous membrane of the accommodating space.

Based on the above, in an embodiment, by controlling the diameter of the pores of the porous membrane and generating the vertical flow field, the RBC debris is separated from WBCs and CTCs. Thus, the purpose of isolating the red blood cells from the blood sample without using centrifugation technology may be achieved, and cells such as WBCs and CTCs are retained completely and the activity thereof is prevented from being damaged.

To make the aforementioned more comprehensible, several embodiments accompanied with drawings are described in detail as follows.

### BRIEF DESCRIPTION OF THE DRAWINGS

The accompanying drawings are included to provide a further understanding of the

disclosure, and are incorporated in and constitute a part of this specification. The drawings illustrate exemplary embodiments of the disclosure and, together with the description, serve to explain the principles of the disclosure.

FIG. 1 is a schematic perspective view of a chip.

FIG. 2A to FIG. 2D are schematic cross-sectional views of a method for isolating red blood cells from a whole blood according to the embodiment of the invention.

### DESCRIPTION OF THE EMBODIMENTS

In order to make the above and other purposes, features, and advantages of the invention more clearly understood, the embodiments of the invention will be exemplified below, and will be described in detail below in conjunction with the accompanying drawings. Furthermore, the direction terms mentioned in the invention, such as “up”, “down”, “front”, “rear”, “left”, “right”, “inside”, “outside” or “lateral side”, etc., just refer to the direction of the additional schema. Accordingly, the terminology of direction is used to describe and understand the invention, not to limit the invention.

The invention integrates a porous membrane and a microchannel therebelow. Thus, the vertical flow field generated by the liquid flowing through the microchannel may drive the RBC debris to penetrate through the porous membrane and thus enter the microchannel, while cells such as WBCs and CTCs are retained on the porous membrane. Accordingly, the purpose of isolating the red blood cells from the blood sample without using centrifugation technology may be achieved, and cells such as WBCs and CTCs are retained completely and the activity thereof is prevented from being damaged.

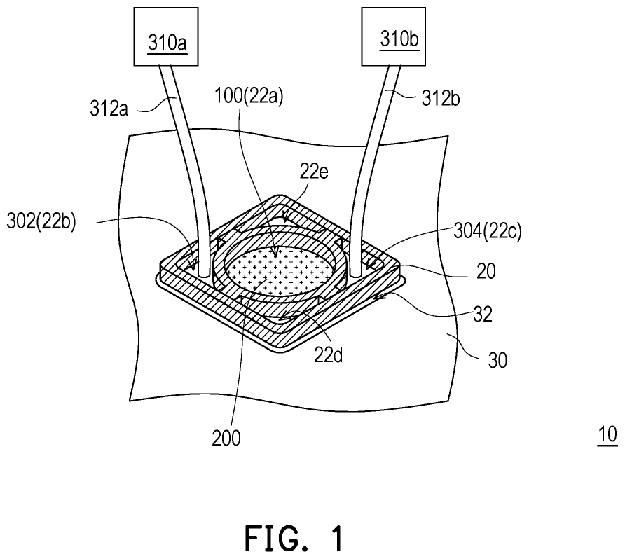

FIG. 1 is a schematic perspective view of a chip.

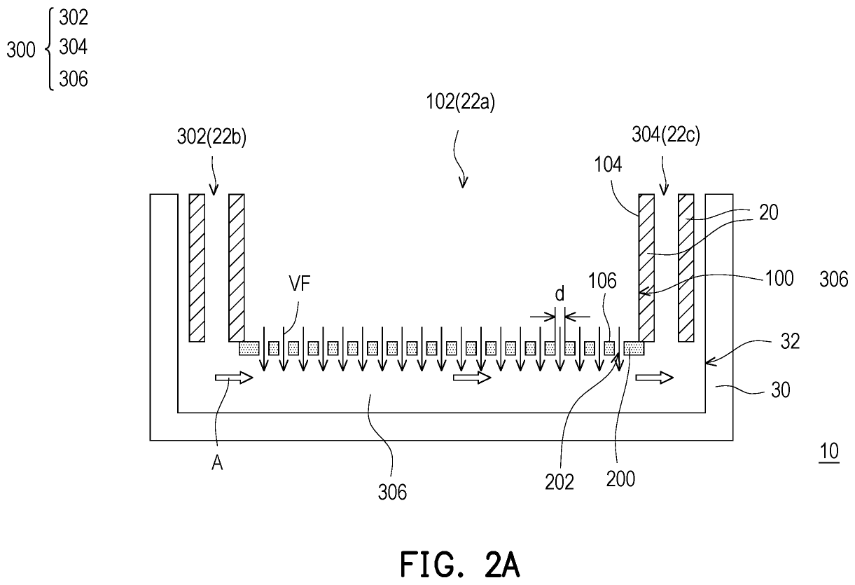

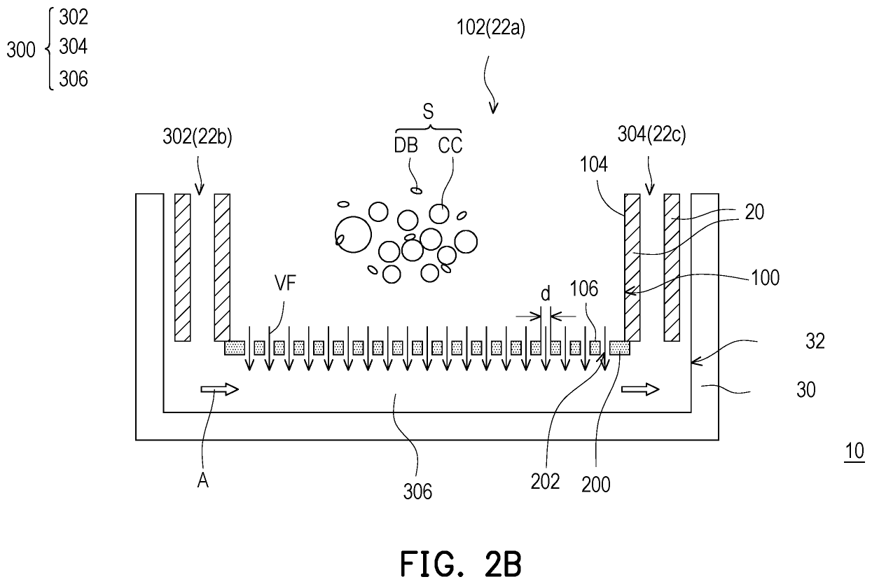

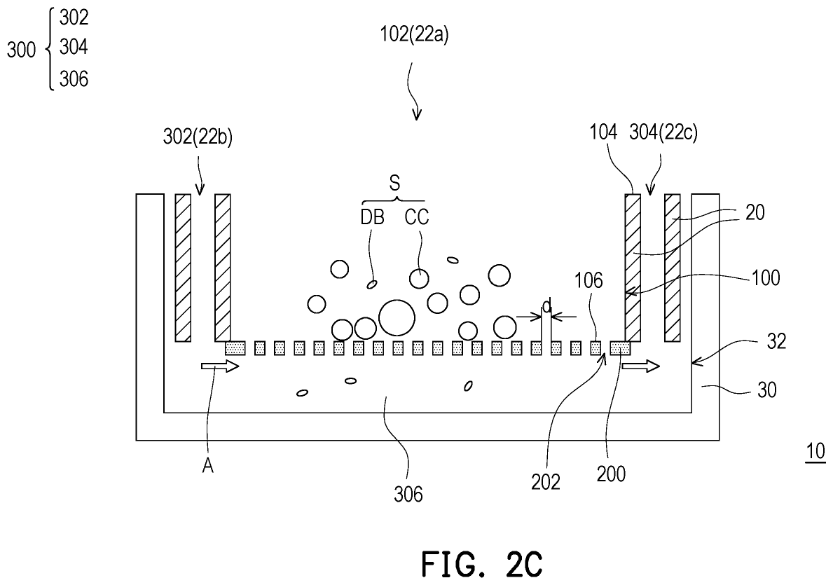

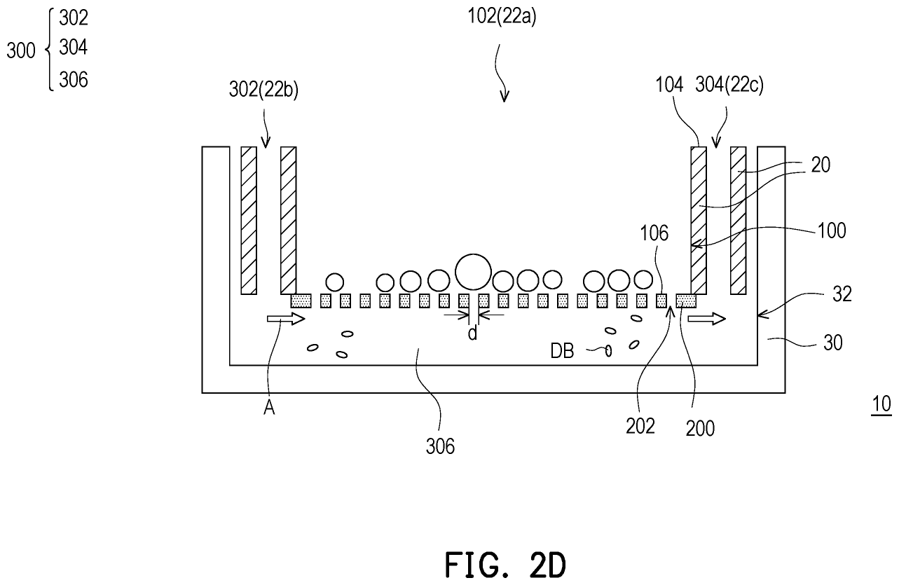

FIG. 2A to FIG. 2D are schematic cross-sectional views of a method for isolating red blood cells from a whole blood according to the embodiment of the invention.

Referring to FIG. 1 and FIG. 2A, the chip 10 includes an accommodating space 100, a porous membrane 200 and a microchannel system 300. The accommodating space 100 is configured to contain a blood sample including RBC debris. The accommodating space 100 is, for example, a tank. The accommodating space 100 has an opening 102, a sidewall 104, and a bottom 106 surrounded by the sidewall 104, so that the accommodating space 100 has a depth. For example, the chip 10 may has the accommodating space 100 formed by the sidewall 104 and the bottom 106. From a top view, as shown in FIG. 2A, the accommodating space 100 is circular, rectangular, or other suitable shape.

The porous membrane 200 is disposed at the bottom 106 of the accommodating space 100 and includes a plurality of pores 202. For example, the porous membrane 200 has a shape corresponding to the bottom 106 of the accommodating space 100, such as a circle. The dimension of the porous membrane 200 is, for example, approximately larger than the dimension of the bottom 106 of the accommodating space 100. In some embodiments, the material of the porous membrane 200 includes polyethylene terephthalate (PET) or other suitable materials. The pore 202 penetrates through the porous membrane 200, and the pore 202 is, for example, circular.

The pores 202 are, for example, regularly arranged. The diameter d of the pore 202 is larger than the size of the RBC debris and smaller than the mean diameter of white blood cells (WBCs) and circulating tumor cells (CTCs). The diameter d of the pore 202 is, for example, less than 8 μm, and is, for example, 5 μm. The depth of the pore 202 is, for example, equal to the thickness of the porous membrane 200, such as 50 μm to 100 μm.

The microchannel system 300 includes a liquid inlet 302, a liquid outlet 304 and a microchannel 306. The microchannel 306 is located between and in communication with the liquid inlet 302 and the liquid outlet 304. In some embodiments, the liquid inlet 302 is configured to receive a liquid and the liquid outlet 304 is used to receive a liquid out of microchannel 306. In some embodiments, the liquid inlet 302 and the liquid outlet 304 are, for example, tanks, respectively. The diameter of the liquid inlet 302 and the liquid outlet 304 is, for example, 1.8 mm to 2.2 mm, and for example, 2 mm. The depth of the liquid inlet 302 and the liquid outlet 304 is, for example, 1.8 mm to 2.2 mm, and for example, 2 mm.

The microchannel 306 is, for example, a tank with depth. In some embodiments, the microchannel 306 is located below the porous membrane 200, the liquid flowing through the microchannel 306 generates a vertical flow field VF, which drives the RBC debris to penetrate through the porous membrane 200 and enter the microchannel 306.

In some embodiments, as shown in FIG. 1 and FIG. 2A, specifically, the chip 10 includes, for example, a lower part 30, an upper part 20, and a porous membrane 200 sandwiched between the upper part 20 and the lower part 30. The lower part 30 is, for example, a main portion of the chip having the tank 32. The material of the lower part 30 may be a high light transmittance material such as glass or plastic. In some embodiments, a top view of the tank 32 is, for example, rectangular. In an embodiment (not shown), the chip 10 includes a multi-well incubation plate, such as a 64-well plate or a 128-well plate. In such embodiment, each well corresponds to the lower part 30 and forms the tank 32, so that the chip 10 includes a plurality of lower parts 30, a plurality of upper parts 20 and a plurality of porous membrane 200 respectively sandwiched between the upper parts 20 and the lower parts 30. The upper parts 20, the lower parts 30, and the porous membrane 200 are respectively arranged in a matrix, for example. Similarly, the chip includes a plurality of tanks 32 arranged in a matrix, for example. In such an embodiment, the sidewall of the tank 32 is, for example, a partition wall between the adjacent wells.

In some embodiments, the porous membrane 200 is placed at the bottom of the tank 32, and then the upper part 20 is placed in the tank 32 on the porous membrane 200. Specifically, the upper part 20 may be engaged with the tank 32 of the lower part 30, for example. The upper part 20 has, for example, a shape corresponding to the tank 32, such as a rectangle. In some embodiments, the upper part 20 has, for example, a plurality of openings 22a-22e separated from each other. For example, the upper part 20 has an opening 22a, openings 22b and 22c on opposite sides of the opening 22a, and openings 22d and 22e on opposite sides of the opening 22a. In some embodiments, the openings 22b-22e are, for example, surrounding the opening 22a, but the invention is not limited thereto. When the upper part 20 is combined with the lower part 30, the openings 22a-22e of the upper part 20 and the tank 32 of the lower part 30 constitute a plurality of separated spaces. For example, the opening 22a of the upper part 20 and the lower part 30 constitute the accommodating space 100, and the openings 22b, 22c and the lower part 30 respectively constitute the liquid inlet 302 and the liquid outlet 304. The opening 22a of the upper part 20 serves as the opening 102 of the accommodating space 100, the sidewall of the opening 22a of the upper part 20 serves as the sidewall 104 of the accommodating space 100, and the upper surface of the porous membrane 200 serves as the bottom 106 of the accommodating space 100. In addition, the tank formed between the porous membrane 200 and the lower part 30 constitutes the microchannel 306. Therefore, when the upper part 20, the porous membrane 200, and the lower part 30 are combined, the accommodating space 100, the porous membrane 200, and the microchannel system 300 are formed, thereby forming the chip 10. In some embodiments, the porous membrane 200 has, for example, a shape corresponding to the opening 22a of the upper part 20, such as a circle. In some embodiments, the chip 10 further includes tubes 312a, 312b, to connect the liquid inlet 302 and the liquid outlet 304 to the pump 310a, 310b, respectively. For example, the two ends of the tube 312a are connected to the pump 310a and the liquid inlet 302, respectively, and the two ends of the tube 312b are connected to the pump 310b and the liquid outlet 304, respectively. In some embodiments, the tube 312a is, for example, connected to a liquid supply bottle (not shown), and the tube 312b is, for example, connected to a waste liquid collection bottle (not shown). The pumps 310a, 310b are, for example, peristaltic pumps.

In some embodiments, the material of the upper part 20 may be a high light transmittance material such as polydimethylsiloxane (PDMS), glass or plastic. The upper part 20 is fabricated by, for example, injection molding, over-molding and drilling processes, etching or other suitable methods. In some embodiments, four openings 22b-22e surrounding the opening 22a is illustrated, however, the invention is not limited thereto. In other words, in other embodiments, numbers, shapes and arrangements of the openings 22a-22e may be adjusted as long as there are two openings at both ends of the other opening.

Next, a method for isolating red blood cells from a whole blood using the chip will be described.

First, a pretreatment is performed on a whole blood sample. In some embodiments, the whole blood sample is mixed with a red blood cell lysis buffer (RBC lysis buffer) to lyse red blood cells into RBC debris DB. The whole blood sample includes, for example, red blood cells and other cells CC, and the cells CC include white blood cells (WBCs), circulating tumor cells (CTCs), and the like. In some embodiments, the whole blood sample and the RBC lysis buffer may be mixed in a volume ratio of 1:2, and allowed to stand for a period of time to react. For example, of the whole blood sample and 1 ml of the RBC lysis buffer are mixed and allowed to stand for 10 minutes. Then, a blood sample S (also referred to as a mixture) including RBC debris DB is obtained. In other words, the blood sample S undergone pretreatment does not substantially include red blood cells. In some embodiments, the pre-treated blood sample S includes RBC debris DB and the cells CC such as WBCs and CTCs. The size of the RBC debris DB is, for example, less than 5 μm, and the mean diameter of the cells CC, such as WBCs and CTCs, is much larger than 5 μm.

Meanwhile, referring to FIG. 2A, the chip 10 is provided, and the microchannel system 300 is turned on to wet the microchannel 306 and generate the vertical flow field VF flowing through the porous membrane 200. Specifically, the pumps 310a, 310b are turned on to supply a the washing liquid A to the liquid inlet 302 through tube 312a, so that the washing liquid A flows through the microchannel 306 into the liquid outlet 304. At the same time, the washing liquid A exiting the liquid outlet 304 is drained through the tube 312b. The washing liquid A may be phosphate buffered saline (Phosphate buffered saline, PBS) or other suitable washing liquid. In some embodiments, the power of the pumps 310a, 310b is adjusted, so that the flow rate difference between the washing liquid A entering the liquid inlet 302 and the washing liquid A exiting the liquid outlet 304 is, for example, greater than or equal to 50 ml/hour, to generate vertical flow field VF. For example, the flow rate of the washing liquid A entering the liquid inlet 302 is, for example, 250 ml/hour, and the flow rate of the washing liquid A exiting the liquid outlet 304 is, for example, 300 ml/hour. The vertical flow field VF is, for example, perpendicular to the surface of the porous membrane 200. Next, referring to FIG. 2B, the pre-treated blood sample (i.e., mixture) S is dropped into

the accommodating space 100 of the chip 10. In some embodiments, the pre-treated blood sample S is dropped into the accommodating space 100 of the chip 10 in multiple times (such as times). The blood sample (i.e., mixture) S to be dropped each time is, for example, 300 fil. As mentioned before, the pre-treated blood sample S includes the RBC debris DB and other cells CC including WBCs and CTCs. In some embodiments, the vertical flow field VF generated by the flow rate difference caused by the pumps 310a and 310b allows the blood sample S continuously pass through the porous membrane 200, so as to achieve the purpose of removing the RBC debris DB from the blood sample S. Specifically, the diameter d of the pore 202 of the porous membrane 200 is larger than the size of the RBC debris DB and smaller than the mean diameter of the cells CC such as WBCs and CTCs. Therefore, as shown in FIG. 2C, under the vertical flow field VF (shown in FIG. 2A and FIG. 2B and omitted in FIG. 2C and FIG. 2D for the sake of clarity of the drawings), gravity and the like, the RBC debris DB will pass through the pores 202 and enter into microchannel 306, and then exits the chip 10 through the liquid outlet 304. In some embodiments, the RBC debris DB may further enter into the waste bottle through the tube 312b. On contrary, since the mean diameter of the cells CC such as WBCs and CTCs is larger than the diameter d of the pore 202 of the porous membrane 200, the cells CC such as WBCs and CTCs are retained on the porous membrane 200. In this way, as shown in FIG. 2D, the RBC debris DB is isolated from other cells CC such as WBCs and CTCs. That is, the red blood cells in the whole blood sample are separated from other cells CC such as WBCs and CTCs. In some embodiments, the pre-treated blood sample (i.e., mixture) S dropped each time is, for example, 300 μl, the flow rate of the washing liquid A entering the liquid inlet 302 is, for example, 250 ml/hour, and the washing liquid exiting the liquid outlet 304 is, for example, 300 ml/hour. In some embodiments, 300 μl of blood sample (i.e., mixture) S may be treated in about 7 minutes, so it takes about 1 hour to treat 0.5 ml of whole blood sample (i.e., 1.5 ml of mixture), and about 300 ml of waste solution is produced. In some embodiments, the blood sample (i.e., mixture) S is initially red due to RBC debris DB, but after removing the RBC debris DB through the chip 10, the blood sample (i.e., mixture) S is substantially colorless.

To sum up, the invention integrates a porous membrane and a microchannel therebelow. Thus, the vertical flow field generated by the liquid flowing through the microchannel may drive the RBC debris to penetrate through the porous membrane and thus enter the microchannel, while cells such as WBCs and CTCs are retained on the porous membrane. Accordingly, the purpose of isolating the red blood cells from the blood sample without using centrifugation technology may be achieved, and cells such as WBCs and CTCs are retained completely and the activity thereof is prevented from being damaged. Therefore, the chip of the invention may be widely used in clinical blood test tools.

It will be apparent to those skilled in the art that various modifications and variations may be made to the disclosed embodiments without departing from the scope or spirit of the disclosure. In view of the foregoing, it is intended that the disclosure covers modifications and variations provided that they fall within the scope of the following claims and their equivalents.

What is claimed is:

1. A chip for isolating red blood cells from a whole blood, comprising:
an accommodating space, configured for containing a whole blood sample including red blood cell (RBC) debris;
a porous membrane, disposed at a bottom of the accommodating space and comprising a plurality of pores, wherein a diameter of the pores is larger than a size of the RBC debris and larger than a mean diameter of white blood cells (WBCs) and circulating tumor cells (CTCs); and
a microchannel system, comprising a liquid inlet, a liquid outlet and a microchannel, wherein the microchannel is disposed between and communicates with the liquid inlet and the liquid outlet, and the microchannel is disposed under the porous membrane.

2. The chip of claim 1, wherein a material of the porous membrane comprises polyethylene terephthalate (PET).

3. The chip of claim 1, wherein the pores of the porous membrane are regularly arranged.

4. The chip of claim 1, wherein the diameter of the pores of the porous membrane is less than 8 um.

5. The chip of claim 1, wherein a liquid flowing through the microchannel generates a vertical flow field, and the vertical flow field drives the RBC debris to penetrate through the porous membrane into the microchannel.

6. The chip of claim 5, wherein the microchannel system further comprises a pump, and the vertical flow field is generated by causing a flow rate difference between a liquid entering the liquid inlet and the liquid exiting the liquid outlet through the pump.

7. A method for isolating red blood cells from a whole blood, comprising:
providing the chip of claim 1;
mixing a blood sample with a red blood cell lysing solution, to lyse red blood cells into RBC debris;
dropping the blood sample including the RBC debris into the accommodating space of the chip; and
providing a washing liquid to the microchannel system of the chip, so that a flow rate difference between the washing liquid entering the liquid inlet and the washing liquid exiting the liquid outlet is generated, and the flow rate difference drives the RBC debris in the blood sample to penetrate through the porous membrane into the microchannel.

8. The method of claim 7, wherein the flow rate difference between the washing liquid entering the liquid inlet and the washing liquid exiting the liquid outlet is greater than or equal to 50 ml/hour.

9. The method of claim 7, wherein the diameter of the pores of the porous membrane is less than 8 um.

10. The method of claim 7, wherein after driving the RBC debris in the blood sample to penetrate through the porous membrane into the microchannel, WBCs and CTCs in the blood sample are retained on the porous membrane of the accommodating space.
